(19) United States
(12) Patent Application Publication
(10) Pub. No.: US 2025/0176861 A1
(43) Pub. Date: Jun. 5, 2025

(54) METHOD FOR DETERMINING A PARAMETER OF THE RESPONSE OF AN AUDITORY NERVE OF A SUBJECT AND ASSOCIATED METHODS AND DEVICES

(71) Applicants: INSTITUT NATIONAL DE LA SANTE ET DE LA RECHERCHE MEDICALE, Paris (FR); UNIVERSITE DE MONTPELLIER, Montpellier (FR)

(72) Inventors: Jean Luc PUEL, Montpellier (FR); Jérôme BOURIEN, Montpellier (FR)

(21) Appl. No.: 18/843,732

(22) PCT Filed: Mar. 7, 2022
(86) PCT No.: PCT/IB2022/000116

§ 371 Date: Sep. 4, 2024

(51) Int. Cl. A61B 5/12; A61B 5/00; A61B 5/388; G16H 50/20

(52) U.S. Cl. CPC A61B 5/125; A61B 5/388; A61B 5/4836; A61B 5/4848; A61B 5/7203; A61B 5/725; G16H 50/20

(57) ABSTRACT

The present invention relies on the need for characterizing the response of the auditory, notably for diagnostic purpose and adjusting of the hearing apparatuses. For this, the inventors have found a new marker, which is the peak-to-plateau ratio of the peri-stimulus time response. The access to this value is easier to obtain than previously known markers. In particular, such value may be obtained in a non-invasive way opening the path to a non-invasive marker of ear disorder. The inventors already have obtained experimental proofs of the relevance of the peak-to-plateau ratio of the peri-stimulus time response in gerbil and in human beings.

### TECHNICAL FIELD OF THE INVENTION

The present invention concerns a method for determining at least one parameter of the response of an auditory nerve of a subject. The present invention also relates to the use of this method for determining in other methods. The present invention also concerns corresponding devices, namely a computer program product and a computer readable medium.

### BACKGROUND OF THE INVENTION

Hearing relies on auditory nerve fibers (ANFs), which convey the neural spike trains initiated by the sensory cells of the cochlea to the cochlear nuclei. ANFs differ in spontaneous spike rate, threshold of activation, and the stimulus level at which the spike rate saturates, and thereby the population of ANFs achieves intensity coding over a large dynamic range.

Beside their spontaneous spike rate and threshold for activation, the ANFs display a typical pattern of discharge in response to a constant sound. At the onset of sound stimulation, the spike rate first increases and reaches an onset peak. The spike rate then declines to a steady-state value (plateau). On cessation of the stimulus, the spike rate drops below the spontaneous rate before gradually recovering.

Given the role of ANFs in sound coding, characterizing ANF populations and their properties is an important approach to studying auditory deficits.

However, electrophysiological tests routinely used in the clinic (i.e., measurements of auditory brain-stem responses) only capture the first spikes of ANFs occurring in synchrony at the onset of the stimulus. Therefore, the assessment of other aspects, such as spontaneous discharge rate and adaptation time constants, requires a single-fiber recording technique, which is too invasive to be achieved in humans and too difficult and time consuming to be routinely conducted in animals.

Another approach relies on the far-field responses from an electrode placed near the cochlea, for example, on the round window membrane, or directly on the auditory nerve, which is much more invasive. In response to a low-frequency pure tone (typically below 2 kHz), the phase-locked responses of ANFs produce an AC component in the gross potential recorded from the nerve, called neurophonic. Interestingly, the neurophonic displays a rapid onset and a response decrement similar in appearance to the adaptation of the firing rate of ANFs.

Nevertheless, when recorded from an electrode placed on the round window membrane, the neurophonic is contaminated by the microphonic potential originated from hair cells, making its measurement difficult. In response to a high-frequency pure tone, the neurophonic is absent because the ANFs do not phase lock and only the compound action potential (CAP) of the auditory nerve that reflects the well-synchronized spikes at the tone-burst onset remains.

### SUMMARY OF THE INVENTION

There is a need for a method enabling to determine a parameter of the response of an auditory nerve in an easier way.

To this end, the specification describes a method for determining at least one parameter of the response of an auditory nerve of a subject, the method being computer-implemented and comprising the following steps:
receiving cochlea response signals, each response signal being the electrical response of a subject's cochlea to a respective excitation, to obtain received response signals,
processing the response signals to obtain a peri-stimulus time response of the auditory nerve, the peri-stimulus time response comprising a peak corresponding to a first amplitude value and a plateau corresponding to a second amplitude value,
calculating the ratio between the first amplitude value and the second amplitude value, to obtain a peak-to-plateau ratio, and
deducing a parameter of the response of the auditory nerve based on the peak-to-plateau ratio.

According to further aspects of the method for determining, which are advantageous but not compulsory, the method for determining might incorporate one or several of the following features, taken in any technically admissible combination:
during the processing step, the received signals are shared into sets of received signals comprising a predefined number of consecutive received signals, preferably two, the processing step comprising applying processing operations on each set of received signals, to obtain a set of processed received signals, and applying an average operation on the set of processed received signals to obtain the peri-stimulus time response of the auditory nerve.
the processing operations comprises a neurophonic isolation operation to obtain a neurophonic potential, and an extracting operation, the extracting operation extracting the temporal envelope of the neurophonic potential.
the neurophonic isolation comprises an averaging sub-operation followed by a filtering sub-operation.
the averaging sub-operation comprises applying a weighted sum.
the filtering sub-operation comprises applying a band-pass filter.
a lower frequency is defined for the band-pass filter, the lower frequency being comprised between 200 Hz and 700 Hz, preferably between 200 Hz and 400 Hz.
an upper frequency is defined for the band-pass filter, the upper frequency being comprised between 1000 Hz and 1500 Hz, preferably between 1100 Hz and 1300 Hz.
the extracting operation comprises applying a rectification sub-operation followed by a smoothing sub-operation.
each excitation is a bandpass-filtered noise.
the method comprises a step for fitting the peri-stimulus time response by two decaying exponential functions to determine the first amplitude value and the second amplitude value.
a parameter determined at the determining step is the spontaneous rate of the fibers of the auditory nerve.

The specification also relates to the use of a method for determining as previously described in any one of the following methods:
a method for predicting that a subject is at risk of suffering from an auditory disorder, the method for predicting at least comprising the step of:
   carrying out the steps of a method determining at least one parameter of the response of an auditory nerve of the subject, to obtain at least one determined parameter, the method for determining being as previously described,
   predicting that the subject is at risk of suffering from the auditory disorder based on the determined parameters;
a method for diagnosing an auditory disorder, the method for diagnosing at least comprising the step of:
   carrying out the steps of a method determining at least one parameter of the response of an auditory nerve of the subject, to obtain at least one determined parameter, the method for determining being as previously described, and
   diagnosing the auditory disorder based on the determined parameters;
a method for identifying a therapeutic target for preventing and/or treating an auditory disorder, the method comprising the steps of:
   carrying out the steps of a method determining at least one parameter of the response of an auditory nerve of the first subject, to obtain at least one first determined parameter, the method for determining being as previously described and the first subject being a subject suffering from the auditory disorder,
   carrying out the steps of a method for determining at least one parameter of the response of an auditory nerve of the second subject, to obtain at least one second determined parameter, the method for determining being as previously described and the second subject being a subject not suffering from the auditory disorder, and
   selecting a therapeutic target based on the comparison of the first and second determined parameters;
a method for identifying a biomarker, the biomarker being a diagnostic biomarker of an auditory disorder, a prognostic biomarker of an auditory disorder or a predictive biomarker in response to the treatment of an auditory disorder, the method comprising the steps of:
   carrying out the steps of a method for determining at least one parameter of the response of an auditory nerve of a first subject, to obtain at least one first determined parameter, the method for determining being as previously described and the first subject being a subject suffering from the auditory disorder,
   carrying out the steps of a method for determining at least one parameter of the response of an auditory nerve of a second subject, to obtain at least one second determined parameter, the method for determining being as previously described and the second subject being a subject not suffering from the auditory disorder,
   selecting a biomarker based on the comparison of the first and second determined parameters;
a method for screening a compound useful as a probiotic, a prebiotic or a medicine, the compound having an effect on a known therapeutical target, for preventing and/or treating an auditory disorder, the method comprising the steps of
   carrying out the steps of a method for determining at least one parameter of the response of an auditory nerve of a first subject, to obtain at least one first determined parameter, the method for determining being as previously described and the first subject being a subject suffering from the auditory disorder and having received the compound,
   carrying out the steps of the method for determining at least one parameter of the response of an auditory nerve of a second subject, to obtain at least one second determined parameter, the method for determining being as previously described and the second subject being a subject suffering from the auditory disorder and not having received the compound,
   selecting a compound based on the comparison of the first and second determined parameters, and
a method for adjusting a hearing aid or a cochlear implant.

The specification also concerns a computer program product comprising instructions for carrying out the steps of a method as previously described when said computer program product is executed on a suitable computer device.

The specification also relates to a computer readable medium having encoded thereon a computer program as previously described.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 is a schematic representation of a device for determining a parameter of the response of an auditory nerve of a subject,

FIG. 2 is a schematic representation of the elements of the peripheral auditory system of a human being,

FIG. 3 is a flowchart showing an example of carrying out of a method for determining a parameter of the response of an auditory nerve,

FIG. 4 is a graph showing an experimental PSTR curve obtained at one step of the method for determining of FIG. 3,

FIG. 5 is a representation of the experimental results obtained in a first experiment when carrying out the method for determining of FIG. 3, and

FIG. 6 is a representation of the experimental results obtained in a second experiment when carrying out the method for determining of FIG. 3.

### DETAILED DESCRIPTION OF THE PREFERRED EMBODIMENTS

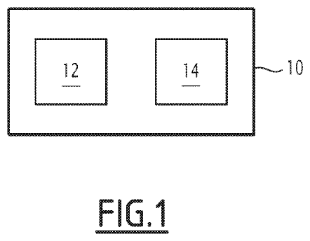

A device for determining 10 at least one parameter of the response of an auditory nerve of a subject is illustrated on FIG. 1.

The device for determining 10 aims at obtaining at least one parameter of the response of an auditory nerve of the subject.

The subject may be a mammal.

In particular, the subject is a rodent such a mouse or a gerbil, or a human being.

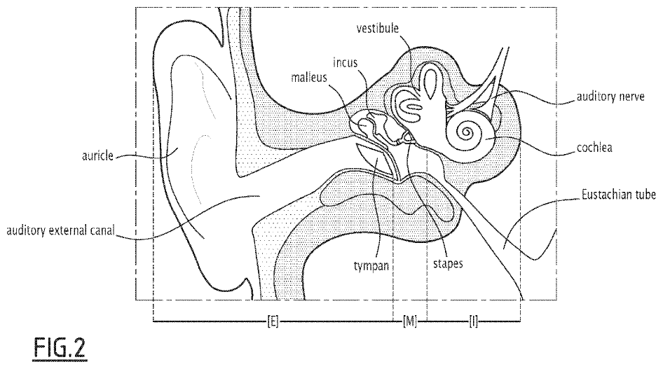

The auditory nerve is part of the subject's ear as apparent on FIG. 2.

FIG. 2 represents schematically the peripheral auditory system allowing the transformation of sound vibrations into a bioelectrical system interpretable by the system central nervous. This system consists of three compartments: the external ear (E), the ear middle (M) and the inner ear (I).

The external ear consists of the auricle and the auditory external canal. It is limited by a flexible membrane, the eardrum, which separates it from the middle ear.

The middle ear comprises a chain of three ossicles: the malleus, the incus and the stapes. The middle ear communicates with the external environment via the Eustachian tube, which ensures the pressure balance of either side of the eardrum.

The inner ear is made up of the vestibule, the organ of balance, and the cochlea, organ of hearing. The cochlea is wrapped in a bony wall, with two openings towards the middle ear: the oval window and the round window. These windows are closed by a membrane, with a permanent contact of the yoke plate on the window membrane oval.

The cochlea is linked to the auditory nerve (sometimes named cochlea nerve) from which the device for determining 10 searches to obtain the response.

The response of the auditory nerve is characterized by many parameters. Each parameters corresponds to a mean parameter of the response of an ANF.

In particular, the spontaneous rate of the fibers of the auditory nerve (SR), which corresponds to the temporal response of the auditory nerve is an example of such parameters.

Another example parameter is the ratio of the number of action potentials generated by the fibers of the auditory nerve (SR) due to the stimulation over the number of action potentials generated by the fibers of the auditory nerve (SR) in the absence of the stimulation.

The device for determining 10 comprises a receiver 12 and a processing unit 14.

The receiver 12 is adapted to receive signals emitted by another apparatus.

For instance, the receiver 12 is an antenna.

In another embodiment, the receiver 12 is adapted to read data in an external memory, when the receiver 12 is in electrical connection with the external memory.

In the present case, the receiver 12 is adapted to obtain cochlea response signals.

The processing unit 14 can be construed as a calculator and comprises a monocore or multicore processor (such as a CPU, a GPU, a microcontroller and a DSP), a programmable logic circuitry (such as an ASIC, a FPGA, a PLD and PLA), a state machine, gated logic or discrete hardware components.

In the present example, the processing unit 14 is adapted to process data, notably by carrying out calculations, and comprises memories adapted to store data and a reader adapted to read a computer readable medium.

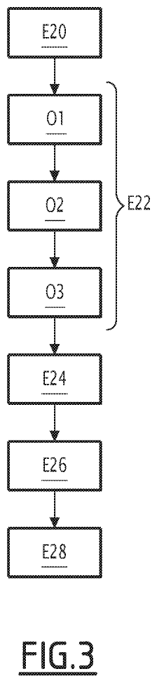

An example of operating of the device for determining 10 is now described in reference to FIG. 3, which illustrates a flowchart of an example of carrying out a method for determining a parameter of the response of an auditory nerve of a subject.

Such method comprises a step for receiving E20, a step for processing E22, a step for determining E24, a step for calculating E26 and a step for deducing E28.

During the step for receiving E20, the receiver 12 receives cochlea response signals.

Each response signal is the electrical response of the cochlea to a respective excitation.

In the present case, the cochlea response signals are the signals recorded on the round window membrane. Therefore, these response signals can be named “round window signals”.

It should be understood that the receiver 12 is not adapted to collect the cochlea response signals, so that the method for determining is a post-processing method: based on a given signal, a measurement relative to the auditory nerve is obtained.

The record of such cochlea response signals is now described.

Such record is achieved thanks to a recording apparatus.

In the present example, the recording apparatus is invasive, such as an electrocochleography device.

However, the recording apparatus may be non-invasive. Notably, the recording apparatus may be a tympanic electrode or alternatively a transtympanic electrode.

In the present example, each excitation is a sound pulse (acoustical bursts).

More specifically, the sound pulse has a duration comprised between 200 ms and 500 ms, preferably comprised between 250 ms and 350 ms.

Each excitation is a bandpass-filtered noise with a trapezoidal envelope and 2.5 ms of rise and fall.

More precisely, each excitation is a 300 ms burst of one-third octave bandpass-filtered noise centred on 4 kHz.

Each excitation is generated by a noise generator adapted to generate random noise by varying different parameters of the excitation such as frequency bandwidth, interstimulus time interval, level, and centre frequency.

In the present case, the excitation are centred on a given frequency, which is the probe frequency, which is comprised between 3 kHz and 5 KHz.

More precisely, each excitation is a 300 ms burst of one-third octave bandpass-filtered noise centred on 4 kHz.

In addition, consecutive excitations are grouped to form a set of consecutive excitations whose response signals will be processed by the same process. The signal to be obtained by the method is the average of the outputs of each of these processes.

In the present case, the number of consecutive excitations in a set is the same and is equal to 2.

Therefore, hereinafter, such set of two consecutive excitations is named a pair.

So as to reduce the cochlear microphonic originating from outer hair cells, the two consecutive excitations has an opposite polarity. By “opposite polarity” in this context, it is to be understood that the phase is inversed between the two consecutive excitations.

In addition, the number of pairs is relative high and the pairs are mutually independent. This enables to obtain a good signal when averaging of the outputs of each process a set of response signals to consecutive excitations.

For example, the number of pairs is comprised between 40 and 60.

The noise generator is commanded to generate mutually independent pairs by refreshing the seed used to generate an excitation when two excitations are generated.

The response of the round-window membrane of the cochlea is recorded by using the recording apparatus positioned on the round-window membrane of the subject.

At the end of the step for receiving E20, the device for determining 10 is provided with the received cochlea response signals.

During the step for processing E22, the processing unit 14 then processes the received signals to obtain a peri-stimulus time response.

The peri-stimulus time response is the temporal response of the auditory nerve of the subject in presence of a stimulus.

More precisely, this peri-stimulus time response is, in the described example, the round-window recorded peri-stimulus time response (PSTR) of ANFs to sound.

The name of peri-stimulus time response is chosen because of its resemblance to the PSTH of fibers.

The PSTH for Peri-Stimulus Time Histogram is an histogram representing the evolution of the discharge rate of a fiber as a function of time. This histogram is constructed from by trains of action potential AP generated by a fiber during iterative stimulation. Before the start of the stimulation, this PSTH represents the basal activity (BA) of the fiber. For stimulation closer in time, this basal activity will tend to be lower than the activity fiber spontaneous. In response to high frequency stimulation, the PSTH of a fiber auditory highlights: a rapid increase in the rate of discharge at the start of stimulation (Peak), followed by a rapid decrease in activity (Adaptation), then a rate of discharge kept constant for the duration of the stimulation (Plateau). At the end of the stimulation, an inhibition of activity is observed, with a rate of discharge which may remain zero for several milliseconds. Thereafter the fiber gradually regains its level of basal activity (Recovery).

The resemblance between PSTR and PSTH comes from the fact that PSTR is characterized by an activated basal voltage of a few μV before stimulation, a synchronization peak at the start of stimulation, adaptation and sustained activity for the duration of the stimulation.

For this, the processing unit 14 applies a series of operation on the received cochlea response signals to obtain the peri-stimulus time response.

In the present case, the series of operation comprises successively a neurophonic isolation operation O1, an extracting operation O2 and an averaging operation O3.

The neurophonic isolation consists in isolating the neurophonic component, which is the response of the ANF to an excitation, from the background noise, which is the response of the ANF in the absence of excitation.

In this example, the processing unit 14 carries out the neurophonic isolation operation O1 by achieving an averaging sub-operation followed by a filtering sub-operation.

The processing unit 14 applies an averaging sub-operation on the two signals of each pair.

The application of the average sub-operation enables to reduce the microphonic potential, which follows the fine structure of the stimulus, while preserving the neurophonic component that stems from the phase-locked activity of ANFs to stimulus envelope fluctuations.

In addition, since each cochlea response signal comprises an electrical signal recorded in absence of excitation and corresponding to uncorrelated background noise, the averaging sub-operation reduces the amplitude of the background noise and improves the neurophonic-to-noise ratio.

The output of such averaging sub-operation is thus a set of averaged pair signals.

As a specific way of carrying out the averaging sub-operation, it is applied a weighted sum applied on each received signals, notably a half-summation of the individual received signals of the pairs.

The processing unit 14 then applies a filtering sub-operation on the set of averaged pair signals.

More precisely, in the present case, during the filtering sub-operation, the processing unit 14 applies a band-pass filtering to the averaged pair signals.

The band-pass filter is adapted to filter an incident electrical signal between a lower frequency and an upper frequency.

The values of the filter bandwidth are chosen in accordance with the spectrum of the spontaneous neural noise, the spectrum depending on the kind of subjects.

Generally, the spontaneous neural noise is centred between 800 Hz and 900 Hz.

In the present case, the lower frequency is comprised between 200 Hz and 700 Hz, preferably between 200 Hz and 400 Hz.

The upper frequency is comprised between 1000 Hz and 1500 Hz, preferably between 1100 Hz and 1300 Hz.

In the present example, the band-pass filter is a 300 Hz to 1200 Hz filter.

The output of such neurophonic isolation operation O1 is thus a set of filtered pair signals.

This set of filtered pair signals corresponds to the neuronal component generated by the auditory nerve, which is often named the neurophonic potential.

The processing unit 14 then applies the extracting operation O2 on the set of filtered pair signals.

The extracting operation O2 aims at extracting the temporal envelope of the neurophonic potential.

In the present example, the extracting operation O2 comprises applying a rectification (rectification sub-operation) followed by a smoothing (smoothing sub-operation), to obtain a set of rectified and smooth pair signals.

The rectification applied in this case is a full-wave rectification.

The smoothing consists in this case to apply a smoothing filter.

The smoothing filter consists in moving a window of a given length and to average the value on the given length.

The given length is comprised between 500 ns and 1.5 μs.

The processing unit 14 therefore obtains a set of rectified and smooth pair signals, which corresponds to a set of temporal envelopes.

The processing unit 14 then applies the averaging operation O3 on the set of rectified and smooth pair signals.

The averaging operation O3 consists in averaging the rectified smoothed pair signals over all the pairs.

The averaging operande is here a weighted sum.

The result of this averaging operation O3 is the PSTR.

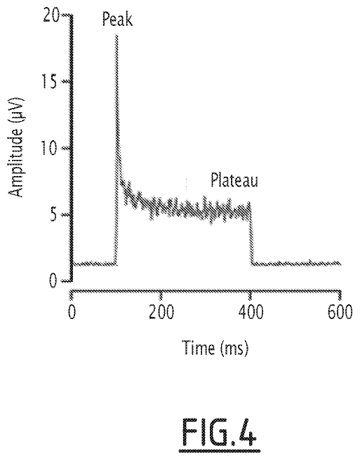

As apparent from FIG. 4, the peri-stimulus time response comprises a peak corresponding to a first amplitude value and a plateau corresponding to a second amplitude value.

During the step for determining E24, the processing unit 14 determines the first amplitude value and the second amplitude value by curve fitting.

For instance, the peri-stimulus time response is fitted by the sum of two decreasing exponential functions with time. Each of these two exponential function has a respective decay time.

In other words, the peri-stimulus time response can be written mathematically as:

$$f ( t ) = F 1 ( t ) + F 2 ( t ) = {Ae}^{- \frac{t}{T 1}} + {Be}^{- \frac{t}{T 2}} + C$$

Where:
F1(t) designates the first decreasing exponential function,
F2(t) designates the second decreasing exponential function,
A, B and C are constants,
T1 is the decay time of the first decreasing exponential function F1(t), and
T2 is the decay time of the second decreasing exponential function F2(t).

The first decreasing exponential function corresponds to the rapid decrease of the peri-stimulus time response while the second decreasing exponential function F2(t) corresponds to the short-term decrease of the peri-stimulus time response added to the steady-state of the peri-stimulus time response.

Another decomposition that may be considered here is a fitting with a model consisting of two exponentially decaying (rapid and short-term) components and a constant, steady-state component. Such decomposition is detailed in the experimental section.

The fitting can be achieved by any technique, and notably a least squares technique.

During the step for calculating E26, the processing unit 14 calculates the ratio between the first amplitude value and the second amplitude value, to obtain a peak-to-plateau ratio.

With the decomposition used in the present example, the first amplitude value (peak) is equal to the sum of A±B±C and the second amplitude value (plateau) is equal to C.

During the step for deducing E28, the processing unit 14 then deduces a parameter of the response of the auditory nerve based on the peak-to-plateau ratio.

For instance, for the SR parameter, the processing unit 14 uses a linear relationship.

For other parameters, it can be imagined that a transfer function is known (by being determined experimentally before) and that the processing unit 14 applies this transfer function to the peak-to-plateau ratio to obtain the value of the searched parameter.

This method therefore enables to provide an access to a parameter of the response of the auditory nerve in an easier way.

Such method is based on using the property of a signal PSTR, which was not used before and more specifically its peak-to-plateau ratio.

This PSTR curve enables to predict adaptation and spontaneous firing of auditory-nerve fiber.

Thus, given the fundamental role of ANFs in sound coding, understanding properties of ANF responses in health, and the relative vulnerabilities of ANF fiber subtypes to injury, is key to studying auditory deficits.

Such method for determining will provide a better understanding of auditory disorders such as neuropathies, tinnitus, and hyperacusis, and will help to improve hearing-aid fittings.

Therefore, such method for determining can be used in a method for predicting that a subject is at risk of suffering from an auditory disorder, a method for diagnosing an auditory disorder, a method for identifying a therapeutic target for preventing and/or treating an auditory disorder, a method for identifying a biomarker, the biomarker being a diagnostic biomarker of an auditory disorder, a prognostic biomarker of an auditory disorder or a predictive biomarker in response to the treatment of an auditory disorder, a method for screening a compound useful as a probiotic, a prebiotic or a medicine, the compound having an effect on a known therapeutical target, for preventing and/or treating an auditory disorder and a method for adjusting a hearing aid or a cochlear implant.

### Experimental Section—Experiment 1

In the experiment described hereinafter, the inventors have proposed an alternative approach based on an electrical signal recorded at the round window in response to a band-pass-filtered noise rather than a pure tone. Round-window response evoked by a bandpass-filtered noise burst displays a synchronized response at the onset of stimulation followed by an AC component arising from a phase-locked activity of ANFs to stimulus envelope fluctuation. After full wave rectification, round window response is characterized by a fast onset peak followed by an adaptation and a steady-state response until the end of the stimulation, mimicking the shape of a peristimulus time histogram (PSTH) from single ANFs.

Because of the similarity of shape with the PSTH of a single ANF, the inventors have assumed that temporal pattern of the peristimulus time responses (PSTRs) recorded at the round window reflects the time constants of adaptation of ANFs during sound stimulation. To address this hypothesis, the inventors performed simultaneous recordings of round-window PSTR and single-fiber PSTH in gerbils.

Indeed, the PSTRs displayed comparable kinetics to those measured from PSTHs derived from single fibers. Additionally, PSTRs nicely predict the rapid time constant and the PSTH peak-to-plateau value of ANFs in gerbils and mice.

Finally, the inventors provide data from gross auditory nerve recordings in humans, supporting the use of PSTRs as a promising tool to better understand auditory-nerve dysfunctions.

### Materials and Methods

### Gerbil and Mouse Experiments

Young adult Mongolian female gerbils and C57BL/6 strain female mice were obtained from Charles River Laboratories. Animals were housed in facilities accredited by the French Ministry of Agriculture and Forestry (Ministère de l′Agriculture et de la Forêt, Agreement C-34-172-36), and the experimental protocol was approved (Authorization CEEA-LR-12111) by the Animal Ethics Committee of Languedoc-Roussillon (France). Experiments were conducted in accordance with the animal welfare guidelines (2010/63/EC) of the European Communities Council Directive regarding the care and use of animals for experimental procedures. All efforts were made to minimize the number and suffering of the animals used.

### Round-Window Recordings

Gerbils and mice were anesthetized by an intraperitoneal injection of a mixture of Rompun 2% (3 mg/kg) and Zoletil 50 (40 mg/kg). The left cochlea was exposed through a retroauricular surgical approach. The recording electrode was placed on the bony edge of the round-window membrane of the cochlea. The bulla (including the recording electrode) was then closed with dental cement.

Electrophysiological recordings were performed in a Faraday-shielded, anechoic, soundproof cage. Animals were placed on a vibration-isolated table (TMC). The rectal temperature was measured with a thermistor probe and maintained at 38° C.±1° C. using a heated blanket beneath the animal. The acoustic stimuli were delivered in closed field under calibrated conditions using a custom acoustic assembly comprising a signal generator (200 kilo samples/s, 24 bit resolution, PXI-4461 controlled by LabVIEW, National Instruments), an audio amplifier (SA1, Tucker Davis Technologies), and a magnetic speaker (MF1, Tucker Davis Technologies) mounted with an adapter and PVC tubing placed in the external meatus.

Electrical signals were recorded in response to acoustical bursts (trapezoidal envelope, 2.5 ms rise and fall, 300 ms duration) of bandpass-filtered noise using different parameters such as frequency bandwidth, interstimulus time interval, level, and center frequency. Two consecutive bursts (called a pair) were presented in opposite polarity to reverse the waveform of the fine structure without changing the waveform of the temporal envelope.

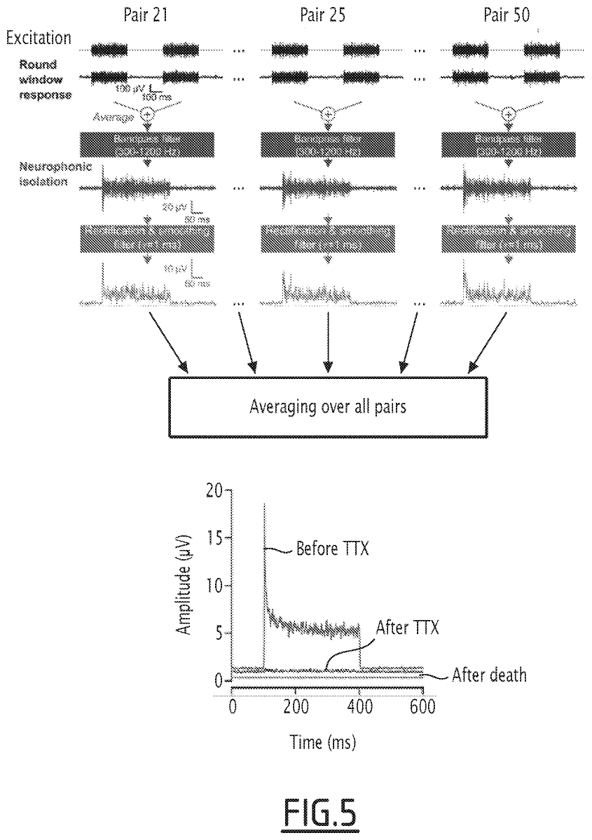

Each pair was designed to be mutually independent by refreshing the seed of the pseudorandom noise generator (see FIG. 5). In total, 50 pairs of burst noise were presented for each sound level and frequency investigated.

The electrical signal was amplified (x 20,000, 1-30,000 Hz filter bandwidth, Grass P511 amplifier) and saved for off-line analysis (50 kilo samples/s, 24-bit analog-to-digital conversion resolution, PXI-4461 controlled by LabVIEW). The two electrical signals within each pair were averaged to reduce the microphonic potential which follows the fine structure of the stimulus, while preserving the neurophonic that stems from the phase-locked activity of ANFs to stimulus envelope fluctuations.

In addition, with the electrical signal recorded during a stimulus-free period (background noise) being uncorrelated, the averaging within each pair reduced the amplitude of the background noise and improved the neurophonic-to-noise ratio.

The averaged electrical signal to each pair was 300-1200 Hz filtered (see FIG. 5). This filter bandwidth was chosen according to the spectrum of the spontaneous neural noise, which is centered: 800-900 Hz in gerbils, mice, and humans.

The filter output was then rectified (full-wave rectification) and smoothed (moving average of the elements of the vector with a fixed window length of 1 ms, function smooth ( ) in MATLAB) to obtain a rectified smoothed signal (see FIG. 5).

Finally, the PSTR was obtained by averaging the rectified smoothed signals over all pairs (see FIG. 5).

### Simultaneous Round-Window and Single Auditory Nerve Fiber Recordings

Simultaneous recordings from the round window and the auditory nerve were only performed in gerbils but not in mice. The round-window electrode was positioned as described above.

Briefly, animals were placed in a custom head holder, and their body temperature was monitored and maintained at 38° C.±1° C.

The calibrated acoustic stimuli were delivered in closed field to the tympanic membrane through magnetic speakers (MF1, Tucker Davis Technologies) coupled to the ear bars. The left cochlear nerve was exposed using a posterior fossa approach. Extracellular action potentials from single auditory nerve fibers were recorded with glass microelectrodes (in vivo resistance between 80 and 110 MV) connected to an AxoClamp 2B (Molecular Devices), filled with 3 M NaCl. A silver-silver chloride reference wire was placed in the neck musculature of the animal. The SR of the fiber (in spikes/s) was estimated by counting the spikes over 30 s. Characteristic frequencies (CFs) of the fibers were measured using a threshold-tracking program (10 spikes/s>SR).

Recordings were obtained in response to the bandpass-filtered noise bursts described above. Simultaneous round-window and single auditory nerve fiber recordings were performed in 43 ANFs. The CF of the fiber, threshold, and SR ranged from 3 and 13.45 KHz, from 2 to 48 dB sound pressure level (SPL), and from 0.6 to 65 spikes/s respectively. Statistical comparison of CF, threshold, and SR distributions from this set of 43 ANFs did not show a significant difference (two-sample Kolmogorov-Smirnov test, p>0.05).

The shape of PSTR and PSTH depends on the temporal resolution on the acquisition system and smoothing filter. A smaller span time makes the response noisy and degrades the quality of the fitting, unless the experimenter increases the number of presentations. Inversely, a larger span time leads to a lack of details to finely evaluate the time course of the response. To be consistent with previously reported data in gerbils, the inventors chose a sampling rate of 50,000 samples/s (i.e., 20 ms resolution) and a smoothing span time of 1 ms for PSTR.

Experimentally, simultaneous recording of PSTR and PSTH is a difficult technique because the recording of a fiber over a long period of time is difficult to achieve. Consequently, the choice of a bin size of 0.5 ms to build the PSTHs was a reasonable compromise between the temporal resolution, the quality of the fitting, and the experimental duration of the acquisition.

Recording from the Auditory Nerve in Humans

The electrophysiological recordings from the auditory nerve in humans were performed in the Reims University Hospital. Patients underwent microvascular decompression to relieve trigeminal neuralgia (n=7) and hemifacial spasm (n=1) via the retrosigmoid approach.

The recordings presented in this experiment were made as part of the routine intraoperative monitoring of auditory-evoked potentials. Such monitoring minimizes the risk of hearing loss resulting from manipulation of the eighth nerve. The Ethics Committee Sud Méditerrannée approved this study. All the subjects (7 females, 1 male) gave their informed consent to participate in this clinical trial. The average age of the patients was 62.1±9.3 years, and they had normal auditory thresholds (<20 dB HL) between 500 Hz and 4000 Hz. Monitoring was based on the CAP of the auditory nerve in response to clicks varying from 0 to 80 dB SPL in 10 dB steps. At the end of the decompression procedure, PSTRs were recorded in response to bursts of one-third octave bandpass-filtered noise (200 ms duration, 2.5 presentations/s, 100 presentations) centered on 4 kHz and presented 40 dB above the click-evoked CAP threshold. It seems that the electrical potential recorded on the surface of the cochlear nerve is weakly contaminated by the microphonic potential originating from cochlear sensory cells and by the neural potential from the cochlear nuclei.

### Data Analysis

Given the shape of the PSTR recorded at the cochlear round window, the inventors used a fitting model to characterize the adaptation of the firing rate in auditory nerve fibers. This fitting model consists of two exponentially decaying components (rapid and short-term) plus a constant term as follows:

$$f ( t ) = {A}_{R} {e}^{- \frac{t}{{τ}_{R}}} + {A}_{ST} {e}^{- \frac{t}{{τ}_{ST}}} + {A}_{SS}$$

Where:
A_{R} is the amplitude and τ_{R} the time constant of the rapid component,
A_{ST} the amplitude and τ_{ST} the time constant of the short-term component, and
A_{SS} the steady-state component reflecting the PSTR plateau

The origin of the time coordinate (t=0) in the fitting model was aligned with the PSTR onset-peak latency detected using the MATLAB max function. Then, baseline mean value measured during the 50 ms preceding the PSTR onset peak was subtracted from the PSTR to remove non-neural and noncochlear electrical contributions.

The fitting was conducted using a nonlinear least-squares procedure (fit function in MATLAB), and five parameters of the PSTR were considered, A_{R}, A_{ST}, A_{SS}, τ_{R} and |_{ST}. The PSTR peak-to-plateau ratio R was calculated as follows:

$$R = \frac{{A}_{R} + {A}_{ST} + {A}_{SS}}{{A}_{SS}}$$

PSTHs were analyzed with the same fitting procedure.

Because of the ANF refractoriness, a silent period frequently appears in the 2 ms following the PSTH onset peak, which produces a very rapid adaptation-like response. To avoid the adaptation-like response distorting the PSTH fitting process, the inventors removed this silent period in the PSTH by interpolation between the onset bin and the first bin following the silent interval.

To test the ability of the PSTR τ_{R} and PSTR peak-to-plateau ratio to respectively predict the PSTH τ_{R} and the ANF SR, the inventors used 222 fibers with a CF between 2.7 and 18 kHz for gerbils and 144 fibers with a CF between 5.5 and 35 kHz for mice.

For statistical analysis, the inventors pooled the fibers in third-octave bins in gerbils and half-octave bins in mice to obtain a minimum of 12 fibers per bin. To measure central tendencies of the population of τ_{R} and SR in each frequency bin, the inventors calculated the arithmetic mean of individual τ_{R} and SR values for each bin. Compared with other population metrics (i.e., median, mode, geometric mean), the arithmetic mean provided the best degree of correlation.

### Statistics

Data are expressed as mean±SEM. Normality of the variables was tested by the Shapiro-Wilks test. If conditions for a parametric test were met, the significance of the group differences was assessed with a one-way ANOVA; once the significance of the group differences (p, 0.05) was established, Tukey's post hoc tests were subsequently used for pairwise comparisons. If conditions were not met, Kruskal-Wallis tests were used to assess the significance of differences among several groups; if the group differences were significant (p,0.05), Dunn's tests were then used for post hoc comparisons between pairs of groups.

### Results

Using electrocochleography in gerbils, the inventors recorded mass potentials at the round window evoked by one-third octave bandpass-filtered noise. A trial consisted of a pair of bursts with opposite polarities to reduce the cochlear microphonic originating primarily from outer hair cells. The response was then filtered at 300-1200 Hz to isolate the neural component. The PSTR was built from the average across trials of a full-wave rectified and smoothed electrical signal.

Consistent with its neuronal origin, acute 30 min round-window infusion of 10 mM tetrodotoxin (TTX) completely abolished the response (see FIG. 5). Note, however, the TTX resistance of the baseline (1.73±0.09 mV before TTX and 1.02±0.05 mV after TTX) and the drop into the noise floor after death (0.30±0.01 mV), suggesting a contribution from some non-neuronal or noncochlear origin.

Therefore, PSTR measurements were referred to the baseline mean value measured before the evoked response.

The PSTR hallmarks consisted of a peak shortly after stimulus onset followed by an adaptation, plus a plateau until the end of the stimulus. Similarly to the PSTH from a single ANF, the PSTR can be well fit as the sum of two exponentially decaying components and a constant, steady-state component (rapid,

$${A}_{R} {e}^{- \frac{t}{{τ}_{R}}} ;$$

short term,

$${A}_{ST} {e}^{- \frac{t}{{τ}_{ST}}} ;$$

steady state, A_{SS}).

Because of the similarity of shape with the PSTH of a single ANF, and its TTX sensitivity, the PSTR likely reflects the activity of fibers within the auditory nerve.

### Characterization of the Peristimulus Time Responses

### Dependence on the Stimulus Bandwidth

In addition to well-synchronized spikes at the onset of stimulation, bandpass-filtered noise drives synchronized spikes that stems from the phase-locked activity of ANFs to stimulus envelope fluctuations. Decreasing the bandwidth of the bandpass-filtered noise reduces envelope fluctuations. Because the plateau of the PSTR relies on envelope fluctuation, the inventors expected a decrease in the amplitude of PSTR plateau with the bandwidth reduction.

To test this hypothesis, the inventors compared PSTRs evoked by noise bursts of different bandwidths to those evoked by pure tones. The center frequency of the noise band was fixed at 4 kHz (i.e., the best sensitivity region of the hearing range of the gerbils), and the level set to 50 dB SPL. Although the PSTR onset peak remained constant across all conditions, reduction of the bandwidth led to an amplitude decrease of the plateau, which, for a pure tone, almost completely disappeared.

A least-squares curve-fitting method was used to quantify the amplitudes and the time constants of the assumed components of the PSTR. The amplitude of the rapid component A_{R} was resistant to the bandwidth reduction and showed a slight non-significant increase (from 9.1±0.8 mV for a half-octave-wide noise burst to 10.6±0.9 mV for a tone burst).

In contrast, the amplitude of the short-term (A_{ST}) and the steady-state (A_{SS}) components decreased for smaller band-widths (A_{ST}, from 1.3±0.12 mV for a half-octave-wide noise burst to 0.12±0.05 mV for a tone burst; A_{SS}, from 2.5±0.27 mV for a half-octave-wide noise burst to 0.25±0.03 mV for a tone burst).

The kinetics of the rapid component became faster with bandwidth reduction (τ_{R}, from 3.3±0.08 ms for a half-octave-wide noise burst to 2.2±0.1 ms for a tone burst), whereas the kinetics of the short-term (τ_{ST}) adaptation component remained insensitive to bandwidth changes.

The PSTR peak-to-plateau ratio increased from 5.4±0.4 for half-octave-wide noise to 17.7±2.5 for one-ninth octave-wide noise. Given that the amplitudes and time constants remained constant down to one-fourth octave bandwidth noise, the inventors chose to use a one-third octave bandwidth, which provides a good compromise between a frequency resolution fine enough to explore different regions of the cochlea and a time duration com-patible with in vivo experiments.

### Dependence on the Interstimulus Time Interval

The ability of the ANFs to respond consistently to repeated acoustic cues depends strongly on the time between successive stimulations. To evaluate the behavior of the PSTRs in this masking phenomenon, the inventors varied the interstimulus interval between two consecutive bursts of one-third octave bandpass-filtered noise centered on 4 kHz from 40 to 600 ms. Prolonging the interstimulus time interval from 40 to 300 ms increased the onset peak amplitude. Above 300 ms, the onset peak remained constant.

The inventors then expressed the amplitude parameters derived from the fits as functions of the interstimulus interval. Like the onset peak, the amplitude of the rapid- and short-term components increased up to 300 ms interstimulus intervals but did not change beyond 300 ms (A_{R}, 4.9±0.5 mV to 9.0±0.8 mV and A_{ST}, 0.5±0.1 mV to 1.5±0.2 mV, from 40 to 300 ms time intervals, respectively).

In contrast, the steady-state component (A_{SS}), which reflects the plateau, was independent of the interstimulus interval. Only the kinetics of the rapid component changed below 300 ms interstimulus interval τ_{R}, 5.7±0.4 ms to 3.3±0.2 ms from 40 to 300 ms time intervals, respectively). Although the amplitude of the short-term component changed with the interstimulus interval, the short-term time constant (τ_{ST}) was unaffected.

Finally, the PSTR peak-to-plateau ratio was stable for silent intervals longer than 200 ms (4.7±0.2). Together, these results are consistent with forward-masking experiments, in which no masking occurs when the time interval between the masker and the signal equals or exceeds 200 ms.

Consequently, the inventors decided to carry out all the experiments using a 300 ms interstimulus time interval.

Simultaneous Recordings of Peristimulus Time Response at the Round Window and of Single Fiber from the Auditory Nerve

To investigate the relationship between the PSTH of single ANFs and the PSTR recorded at the round window, the inventors conducted 43 simultaneous measurements using a sharp glass pipette in the gerbil auditory nerve and a silver ball electrode in the round window niche, respectively. For each ANF, the SR and the CF of the fibers were determined. Both PSTR and PSTH were obtained in response to one-third octave bandpass-filtered noise centered on the CF of the fiber.

### Dependence on Sound-Pressure Level

The inventors first correlated the activity of several ANFs to the corresponding PSTR by comparing the fits of the mean PSTR and the mean PSTH, as a function of sound level. To do so, the inventors selected 17 fibers for which a complete experimental protocol was available, that is, successful recording of activity from 20 to 60 dB SPL.

Although both the mean PSTR and the mean PSTH behaved similarly, amplitude-fitting parameters derived from the mean PSTH tended to saturate for supra-threshold levels (especially for A_{ST} and A_{SS}), whereas those of the mean PSTR increased more slowly and did not saturate, probably because of cochlear spread of excitation.

Plotting the amplitude-fitting parameters of the mean PSTR (A_{R}, A_{ST}, and A_{SS}) against those of the mean PSTH displayed relationships that can be approximated by power-law function for A_{R} (R²=0.99) and a power-law function plus a deviation term for A_{ST} and A_{SS} R²=0.99).

The rapid time constant τ_{R} decreased with increasing sound level, but the short-term time constant τ_{ST} was insensitive to sound level. A power-law function was used to fit the relationship between the mean PSTH ER and the mean PSTR τ_{R} (R²=0.87).

Because the mean PSTH τ_{ST} was independent of the stimulation level (about 50 ms duration), no significant relationship was found between τ_{ST} derived from the mean PSTR and the mean PSTH. Finally, the relationship between the mean PSTH and the mean PSTR peak-to-plateau ratio can be approximated by a power-law function (R²=0.86).

### Dependence on Center Frequency

In the gerbil, inner hair cells (IHCs) from the apical half of the cochlea are mainly innervated by high-SR ANFs, whereas basal IHCs are innervated by ANFs with a greater SR diversity. When measured at a constant level above the threshold (i.e., 30 or 40 dB SL), high-SR fibers have been shown to exhibit a shorter IR and larger PSTH peak-to-plateau ratio than the low-SR fibers.

To test the ability of PSTR τ_{R} and PSTR peak-to-plateau ratio to predict PSTH IR and ANF SR, respectively, the inventors used their simultaneous data recorded at 50 dB SPL. In gerbils, the PSTR threshold is close to 10 dB SPL between 3 and 12.6 KHz.

Thus, a stimulation at 50 db SPL corresponds to 40 dB above the PSTR threshold. The 43 simultaneous recordings were pooled according to the CF of the fiber (3.15-12.6 kHz, in one-third octave steps).

The shape of mean PSTR and mean PSTH computed for fibers pooled per one-third octave band behaved similarly across frequencies. Worthy of note is the pronounced recovery from adaptation after stimulus offset in response to low-frequency stimulation in both mean PSTR and mean PSTH, which fits with a response dominated by high-SR fibers.

A power law was used to fit the relationship between mean PSTH τ_{R} and mean PSTR τ_{R}(y=1.05x^{0.93} with R²=0.99). The rapid time constant τ_{R} was almost the same using both techniques.

A positive correlation was also found between the mean PSTH and the mean PSTR peak-to-plateau ratio (y=1.7x^{0.48} with R²=0.97).

Finally, the inventors investigated the relationship between the mean PSTH peak-to-plateau ratio and the mean SR and the relationship between the mean PSTR peak-to-plateau ratio and the mean SR, when ANFs were pooled according to their CF. Here again, positive relationships (R²=0.96 and R²=0.95) were found. The larger the mean PSTR peak-to-plateau ratio was, the higher the mean SR of the fibers tuned at PSTR probe frequency (y=2.7x^{1.44}).

### Predictive Modeling

Next, the inventors probed whether the power laws used to describe the relationships between PSTR τ_{R} and PSTH τ_{R} and between PSTR peak-to-plateau ratio and mean SR constitute predictive models to infer PSTH τ_{R} and mean SR by measuring PSTR IR and PSTR peak-to-plateau ratio, respectively.

The inventors performed additional recordings in two independent groups of animals. In a first group, the inventors only measured PSTR at the round window. PSTR τ_{R} measurements were used as the input of the previous power-law relationships to compute a prediction interval for PSTH τ_{R}.

Similarly, PSTR peak-to-plateau ratio was used as the input of the power-law relationship and to calculate prediction intervals for mean SR. (Both prediction models are referred to as PSTR-based prediction hereinafter.)

In a second group, the inventors only measured PSTH τ_{R} and mean SR using single-fiber recordings to evaluate the PSTR-based prediction.

### Validation of the Predictive Model in Gerbils

The inventors first measured PSTRs in the independent group of gerbils. The center frequency was varied from 3.15 to 16 KHz in one-third octave step increments, and the noise bursts were presented at 50 db SPL (i.e., 40 dB above the PSTR thresholds).

Note that the A_{R} decreased significantly with frequency from 8.1±0.9 mV for 3.15 KHz to 2.3±0.3 mV for 16 KHz. No significant change was observed for PSTR A_{ST} and A_{SS}.

Although τ_{R} was in the range of simultaneous recording values, its increase with frequency was not statistically significant. No significant change with frequency was also seen for τ_{ST}.

Finally, the frequency dependence of A_{R} leads to a reduction of the PSTR peak-to-plateau ratio from 6.2±0.5 for 3.15 kHz to 3.5±0.2 for 16 KHz.

Next, single-fiber recordings obtained from a previous experiment was reanalyzed to extract PSTH τ_{R} in responses to 50 dB SPL tone bursts (1 ms rise and fall, 50 ms duration, 10 bursts/s) and to evaluate their SR.

The mean PSTH IR was calculated by pooling individual PSTH τ_{R} per one-third octave band according to the CF of the fiber. PSTR-based prediction was derived from PSTR τ_{R} values obtained previously. The mean PSTH τ_{R} falls within the prediction range. The measured PSTH τ_{R} of ANFs and the τ_{R} PSTR-based prediction shows a high degree of correlation (R²=0.9).

Similarly, the inventors plotted the SR of individual ANFs as a function of the CF of the fiber, and the inventors calculated the mean SR from individual SR values pooled per one-third octave band according to CF. Here again, the mean SR matches the PSTR-based prediction derived from PSTR peak-to-plateau ratio, with a strong correlation (R²=0.96).

### Validation of the Predictive Model in Mice

To validate the PSTR as a neural index to infer rapid adaptation time constant and mean SR of a subpopulation of ANFs in another animal model, the inventors recorded PSTRs in C57BL/6 mice. The center frequency was varied from 5.7 to 32 KHz in one-half octave increments, and the noise bursts were presented at 40 dB above the C57BL/6 PSTR thresholds (data not shown).

The amplitude of the rapid component was independent of the probe frequency, as were the short-term and the steady-state components. However, the rapid time constant τ_{R} slightly, but not significantly, increased with the stimulation frequency from 3.1±0.4 ms at 5.7 kHz to 4.1±0.3 ms at 32 kHz. As also shown in gerbils, τ_{ST} was independent of the probe frequency (40.6±2.8 ms across frequency).

Together with the lack of change in the PSTR peak-to-plateau ratio, PSTR characteristics suggest a more homogeneous distribution of the ANFs according to their SR along the tonotopic axis in mice than in gerbils.

To validate this hypothesis, the inventors reanalyzed the single-fiber recordings from the auditory nerve of mice in response to tone bursts (1 ms rise and fall, 50 ms duration, 10 bursts/s) made previously. Because of the small number of recordings in C57BL/6 and lack of striking differences between CBA/CaJ and C57BL/6 mice, the inventors pooled the single-fiber data of both strains.

The criteria of selection were a fiber CF in the range of the PSTR probe frequency (5.7-32 kHz) and the PSTH measured at 40 dB above mean C57BL/6 PSTR thresholds. Based on these criteria, the inventors selected 144 fibers among the 254 recorded in this previous experience.

The mean PSTH τ_{R} slightly, but not significantly, increased with frequency. When the inventors plotted the PSTR-based prediction derived from PSTH τ_{R} in gerbils (R²=0.89), the mean PSTH τ_{R} fell within the prediction range. Also, the mean fiber SR calculated from 144 ANFs remained stable across frequency and fell within the PSTR-based prediction. Although the coefficient of determination was low (R²=0.1), the SR values are distributed along the y=x line.

Recordings from the Auditory Nerve in Humans

For obvious ethical reasons, single-fiber recordings from the cochlear nerve have never been performed in humans, creating a critical gap in the understanding of the applicability of data from animal models.

The inventors took advantage of cerebellopontine angle surgeries to record PSTRs from an electrode placed on the intracranial portion of the cochlear nerve in eight consenting patients (62.1±9.3 years old) with normal hearing (average thresholds between 0.5 and 4 KHz, 12.9±0.9 dB HL). Those patients were undergoing neurosurgeries for cranial-nerve functional disorders (trigeminal neuralgia and hemifacial spasm).

Click-evoked CAPs were recorded intraoperatively to ensure that no noticeable changes occurred in auditory nerve function as a result of the surgical dissection). Then PSTRs were recorded in response to noise bursts centered on 4 kHz, presented 40 dB above the click-evoked CAP threshold (i.e., 70 dB SPL).

PSTRs were also obtained using stimulus pairs of opposite polarity. The waveform of the human PSTR was similar to those recorded from gerbils or mice (FIG. 10B). The rapid time constant τ_{R} was equal to 2.7±0.07 ms, which is in the same range as the animal data.

Although the amplitudes of the rapid (A_{R}, 2.0±0.3 mV), short-term (A_{ST}, 0.1±0.1 mV). and steady-state components (A_{SS}, 0.75±0.08 mV) were smaller than of those measured in gerbils in responses to 50 dB SPL noise bursts centered on 4 kHz (i.e., 40 dB SL), the PSTR peak-to-plateau ratio was in the same range (i.e., 5.0±0.1).

By using PSTR-based predictive models identified in gerbils, it can be inferred that the ANFs tuned at about 4 kHz in the human cochlea display a rapid adaptation time constant of 2.7±0.1 ms a mean SR of 22±3 spikes/s.

Together, these results suggest that the PSTR constitutes a promising tool to extract the kinetics of neural adaptation to sound stimulation and to map the SR-based composition of the ANFs in humans and other mammalian species.

### DISCUSSION

Using far-field recordings in rodents, the inventors probed the adaptation kinetics and spontaneous action-potential firing in limited populations of ANFs as a function of their tuning frequency.

In addition, the inventors examined whether the relationship between the PSTR and PSTH allows the prediction of these two electrophysiological features in normal-hearing human ANFs.

### Adaptation of Auditory Nerve Fibers

In response to the onset of an acoustic stimulus, the spike rate of an ANF rapidly increases to a maximum value and thereafter adapts over the course of minutes. The post-onset reduction in discharge rate has been mainly attributed to the reduction of vesicle release at the IHCs ribbon synapse.

Other mechanisms, including the desensitization of postsynaptic glutamate receptors and refractoriness of action potential generation at the ANF level, may also contribute.

The most definitive experimental procedure to investigate ANF adaptation relies on single-fiber recording. However, this approach is very invasive and difficult to achieve as the auditory nerve is deep beneath the cerebellum and largely surrounded by the petrous portion of the temporal bone.

Here, the inventors investigated whether far-field PSTRs recorded at the round window mimic single-fiber PSTHs, especially with respect to the rapid and short-term time constants of the postonset adaptation. With increasing sound level, the inventors saw a reduction of the rapid time constant.

Consistent with this, it has been reported a decrease of the PSTH rapid time constant from 10 to 1 ms as tone level increased, which is in the range of these PSTR data.

In addition, the short-term time constant reported in literature was independent on the stimulus level, and approximated 50 ms, which is also in agreement with these PSTR measurements.

To most directly evaluate how a population of ANFs contributes to the PSTR, the inventors conducted simultaneous recordings of single ANFs and round-window PSTRs. Pair-by-pair analysis showed many similarities, such as the shape and the time constants. Few discrepancies are, however, inherent to the nature of the recording techniques.

For example, PSTH amplitudes (especially A_{ST} and A_{SS}) tend to saturate at suprathreshold levels, whereas those of PSTR continue to increase. Because PSTHs reflect the time course of the discharge rate in response to sound, their amplitudes are limited by the maximum discharge rate of the fiber and the relatively limited dynamic range of single ANFs.

In contrast, the PSTR amplitude can continue to grow for sound levels above 50 dB SPL because higher levels of stimulation recruit ANFs with characteristic frequencies outside the nominal bandwidth of the noise. Thus, at least within a range of 40 dB above threshold, PSTRs may reflect the rapid and short-term adaptation of the single ANFs with CFs that lie within the noise bandwidth.

### Spontaneous Activity of Auditory Nerve Fibers

Several studies have described the SR patterns of ANFs in the gerbil cochlea, with a majority of high-SR fibers in the apical part and a more balanced distribution of high-, medium-, and low-SR fibers in the basal half.

As shown in the present experiment, high-SR fibers exhibit greater peak-to-plateau ratios than low-SR fibers. This is also true in cats, chinchillas, and mice.

Validation of these PSTR-based predictions comes from single-fiber recordings. In gerbils, a correlation was seen between the mean PSTR and the mean PSTH rapid adaptation τ_{R}. For both PSTR and PSTH, the rapid time constant slightly, but not significantly, increased with frequency, which is consistent with single-fiber measurements.

The present experiment also shows a positive correlation between the mean PSTR peak-to-plateau ratio and the mean SR of fibers tuned at the probe frequency. Indeed, many metrics could be used to assess the central tendency of the SR-based population.

Therefore, the inventors compared the degree of correlation between predicted and measured values using arithmetic mean, the median, and the geometric mean. In all cases, the arithmetic mean provides the best degree of correlation (data shown) and thus the best predictor for the mean SR.

Like the PSTR, the mean PSTH in response to 50 dB SPL sound stimulation reflects the activation of an ensemble of single ANFs having different thresholds. The main difference was however that the PSTH was built in response to tone burst, and the prediction was based on responses to band-pass-filtered noise.

This is an interesting point because it is possible to probe the present predictive modeling in other species in which the PSTH has been recorded in response to tone burst (which is generally the case).

Indeed, when recorded on the cochlear round window of mice, the PSTR rapid adaptation τ_{R} and the peak-to-plateau ratio were mostly invariant across frequency, which is consistent with a homogeneous distribution of the SR fibers along the tonotopic axis of the mouse cochlea (mean SR; 30 spikes/s from 5.7 to 32 kHz).

It can thus be concluded that far-field PSTRs may constitute a powerful and less invasive tool to rapidly extract key features of the spontaneous and sound-evoked responses of the auditory nerve in animal models, including mice, in which single-fiber recordings are difficult to achieve.

### Toward a Diagnostic Tool

To date, the only functional data on the human ANFs come from recordings of CAP or wave I of the auditory brainstem responses, which reflect the synchronized activity at stimulus onset.

For ethical reasons (mainly the need to penetrate the auditory nerve with micro-electrodes), single-fiber recording from the auditory nerve is not feasible in humans, making the SR-based composition impossible to investigate.

To collect more data, a non-invasive technique is thus needed, especially to record PSTRs in awake subjects. Given that the spontaneous neural noise (900 Hz peak) can be extracted in the human using ear canal recording techniques, it can be considered that the signal-to-noise ratio is sufficient to extract PSTR using a tympanic electrode or alternatively by further using a transtympanic electrode.

To further investigate the weighted contribution of each pool of ANFs to the PSTR, and to predict their behavior under pathoogic conditions such as ANF loss and/or hair-cell loss, it would be favourable to develop a mathematical model of the human cochlea. This should be based on the morphologic observations.

As a conclusion, PSTRs are a powerful diagnostic tool to capture information on auditory nerve survival and, importantly, SR-based function and dysfunction in humans, providing a better understanding of auditory neuropathies, tinnitus, and hyperacusis.

### Experimental Section—Experiment 2

Here, the inventors established a gerbil model of temporary noise-induced threshold shift with cochlear synaptopathy. In gerbil, the SR-based distribution of ANFs is well characterized. It varies as a function of cochlear location and displays a higher proportion of low-SR fibers than in mouse. This specificity makes the gerbil a useful model to study different SR-based pools of ANFs within the same cochlea for their vulnerability to noise.

The inventors recorded distortion product otoacoustic emissions (DPOAE) to evaluate the functional integrity of the OHCs and CAP of the auditory nerve to assess ANF firing synchrony at the onset of the acoustic stimulation. This electrophysiological approach also included the recording of ensemble ANF spontaneous activity at the level of the cochlear round window and sound-evoked PSTRs to assess their sensitivity to synapse loss and their ability to reflect the relative vulnerability of low-vs. high-SR fibers to noise.

### Materials and Methods

### Animals and Groups

Mongolian gerbils (Meriones unguiculatus) of both sexes were used for all experiments. Animals were born and housed in a colony from breeders obtained from Charles River Laboratories. At age 14 (±5%) weeks (wk) gerbils were noise-exposed and assigned to groups to be tested at various post-exposure times (24 h, 2 wk, or 4 wk after noise). Age-matched, unexposed animals otherwise held identically served as controls. All procedures were approved by the Institutional Animal Care and Use Committee of the Massachusetts Eye and Ear.

### Noise Exposure

Awake gerbils were placed, singly and unrestrained, in a small wire mesh cage suspended directly below the acoustic horn of a sound delivery loudspeaker that extended into a reverberant exposure chamber. A one-octave band of noise (2.8-5.6 kHz) was delivered at 100 db SPL for 2 h. Calibration to the target level was accomplished immediately preceding each exposure session. Sound levels at different locations within the holding cage varied within 1 dB of the target level.

### Physiology

Physiologic testing was conducted in an acoustically and electrically shielded chamber heated to 34° C. Gerbils were anesthetized with ketamine (100 mg/kg ip) and xylazine (5 mg/kg ip). Anesthesia was maintained with periodic administration of ketamine (33-50 mg/kg ip). Heart rate, temperature, and oxygen saturation were monitored throughout testing. A National Instruments PXI-based system with 24-bit digital input/output boards generated all stimuli and captured all responses, controlled by custom LabVIEW-based software. Signals were delivered using a custom, closed acoustic assembly comprising two miniature sound delivery speakers (CDMG15008-03A, CUI) and a detection microphone (FG-23329-PO7) to measure sound pressure in the ear canal. Responses were amplified (10,000×; Grass P511) with a 10-3,000 Hz (CAP) or 3-10,000 Hz (PSTR) pass band. The left ear of each animal was tested.

### DPOAE

Distortion product otoacoustic emissions were elicited with stimuli consisting of two pure tones (f1 and f2) presented at frequencies defined by f2/f1=1.2 and at levels defined by L1=L2±10 dB. Captured from ear canal pressure measurements, DPOAEs of the frequency 2f1−f2 were recorded as functions of increasing stimulus level (L2=0-80 dB SPL, 5 dB steps) at 10 f2 frequencies from 2 to 44 kHz. From the growth functions, iso-response curves were interpolated to determine DPOAE thresholds, defined as the minimum level required to elicit a DPOAE of −5 dB SPL.

### CAP

CAP of the auditory nerve were recorded using a wire recording electrode (platinum-iridium) placed at the round window niche, with subdermal needle electrodes at the vertex (reference) and tail base (ground). CAPs were elicited by tone pips (0.5 ms rise-fall, 5 ms plateau, 16/s). Stimulus frequencies matched DPOAE f2 values, and the level was increased in 5 dB steps from below threshold to 90 dB SPL. Opposite-polarity stimulus pairs (128 tone pips/polarity) were presented for each frequency-level combination. Responses were amplified (10,000×), filtered (10-3,000 Hz), and averaged. Offline, peaks corresponding to N1 and P1 of the action potential were identified visually from stacked waveforms, aided by custom software. The threshold was defined as the lowest level at which repeatable response peaks were evident, and peak-to-peak values of the N1-P1 components were used to calculate response amplitudes.

### Round Window Noise

Electrical activity from the round window in the unstimulated condition (e.g., round window “noise”) was recorded with the same electrode used for CAP assessment. The detected activity was captured over 40 s, amplified (10,000×), and its overall power spectral density (PSD) was estimated using Welch's method (pwelch function using MATLAB language. 2,048 samples per segment, 0% overlapped, rectangular window, sampling rate 100,000 samples/s). The coordinate (frequency, x-axis; amplitude, y-axis) of the spectral peak occurring in the 900 Hz range of the PSD was detected using the max MATLAB function (search window 300-1,200 Hz). The amplitude of the 900-Hz component in the round window noise was also estimated by applying a bandpass filter (300-1,200 Hz, 2nd-order Butterworth filter) to the 40 s trace and calculating the overall root-mean-square (RMS) level. Spectral peak amplitude, frequency, and RMS level were compared across groups of noise-exposed animals and controls.

### PSTRs

Peri-stimulus time responses were elicited using ⅓ octave band noise bursts (trapezoidal envelope, 200 ms duration, 1 ms rise/fall) with center frequencies at each of the 10 CAP test frequencies, levels from 0 to 80 dB SPL in 10 dB steps, and 50 presentations per frequency-level combination. Each “presentation” comprised a pair of bursts presented in opposite polarities to minimize the hair cell-based cochlear microphonic. The seed of the pseudorandom noise generation was refreshed at the first burst of each pair to ensure independence of the stimulus waveform across presentations. Half sums from each presentation pair were filtered (300-1,200 Hz) and the temporal envelope extracted by full-wave rectification and smoothing (1-ms time span). PSTRs were then obtained by averaging the resulting signals. The onset-peak amplitude of the PSTR was estimated using the max MATLAB function, during the first 6 ms of the response. The plateau amplitude of the PSTR was measured by averaging the PSTR samples during the last 50 ms of the response.

### Immunostaining of Cochlear Whole Mounts

Immediately following the testing, subsets of animals from each group were transcardially perfused with 4% paraformaldehyde in 0.1 M phosphate buffer, followed by intralabyrinthine perfusion of fixative through the oval and round windows. Cochleae were post fixed for 2 h at room temperature and decalcified in 0.12 M EDTA for 72 h. The left (tested) cochlea was processed for these studies. The organ of Corti was microdissected into nine pieces, transferred to a sucrose solution (30% sucrose in PBS), permeabilized by freeze/thawing, and blocked in 5% normal horse serum with 0.3% Triton X-100 in PBS for 1 h. Pieces were incubated for ˜20 h at 37.C with primary antibodies then incubated for 2 h at 37° C. with secondary antibodies. All antibodies were diluted in 1% normal horse serum with 0.3% Triton X-100 in PBS. IHC bodies were labeled with an antibody against myosin Vila, a component of hair cell stereocilia and cytoplasm (rabbit anti-myosin Vila, Proteus Biosciences, 1:200; AlexaFluor 647 donkey anti-rabbit, 1:200). Presynaptic ribbons were labeled with an antibody against a predominant ribbon component, C-terminal binding protein 2 (mouse IgG1 anti-CtBP2, BD Biosciences, 1:200; AF 568 goat anti-mouse IgG1, 1:1,000). Post-synaptic glutamate patches were labeled with an antibody against the GluR2 subunit of AMPA-selective glutamate receptors (mouse IgG2a anti-GluA2, Millipore, 1:2,000; Alexa Fluor 488 goat anti-mouse IgG2a, 1:1,000). Cochlear segments were mounted in Vectashield (Vector Laboratories) on a glass microscope slide, arranged from apex to base.

### Hair Cell and Synapse Quantification

Immunostained cochlear segments were imaged at low power (Leica DM5500 epifluorescence microscope, 10× air objective, N.A. 0.4) for quantification of inner and outer hair cell loss. A cochlear frequency map was produced from the same images for each organ of Corti using a custom plug-in for Image J, based on the place-frequency map for gerbil (Müller, 1996). For synapse quantification, confocal z-stacks were acquired (Leica TCS SP5) using a glycerol-immersion objective (63×, N.A. 1.3) and 3.17× digital zoom. The x-y dimensions were fixed for all stacks at 1,024×512 pixels. The y dimension included and extended slightly beyond the tectorial-to-basilar membrane length of IHCs. The z dimension was selected manually for each stack to capture the full modiolar-to-pillar extent of every IHC in the x-y frame. Approximately 15-17 IHCs were imaged at each frequency location by acquiring two adjacent z stacks (0.33 μm spacing). Image stacks were imported to Amira (ThermoFisher Scientific) to quantify hair cells, pre-synaptic ribbons, and post-synaptic glutamate receptor patches. IHCs were inspected for overall morphology based on their myosin-stained cell bodies and quantified based on their CtBP2-stained nuclei. In Amira, a 3D representation of each stack was produced and rotated during quantification to avoid undercounting ribbons obstructed by each other at certain viewing angles. Synapses were quantified as paired pre-synaptic ribbon/post-synaptic glutamate receptor patch puncta at seven cochlear locations from 0.5 to 32 KHz.

### Results

Although this second experiment leads to obtaining very interesting results notably for CAP or DPOAE, only the results linked to PSTR are exposed hereinafter (as was the case for the first experiment).

Peri-stimulus firing adaptation is an important feature of ANF response, largely determined by the IHC-ribbon synapse machinery. PSTHs of single ANF responses to sound reveal a peak of activity at the onset of stimulation followed by adaptation to a steady-state firing plateau that persists with continued stimulation. These features of the PSTH are remarkably well-preserved in the global ANF electrical activity accessible at the level of the cochlear round window, the peri-stimulus time response, PSTR.

Here, with the same RW electrode used to record the CAP and the spontaneous round window activity, the inventors recorded PSTRs evoked by a train of narrow-band noise bursts centered at the probe frequencies used to elicit the CAP. To avoid an excessive spread of excitation, the inventors adjusted the sound level at 30 dB above the threshold, which has been shown to be sufficient to recruit low-SR fibers in the gerbil.

The inventors considered several characteristics of the PSTR recorded 2 and 4 wk after moderate noise exposure, as compared to unexposed controls. At these post-exposure times, PSTR thresholds and DPOAE and CAP thresholds were not different from control levels; thus, noise-induced shifts and OHC dysfunction are not confounded to interpretation.

Two frequencies were selected for comparison; 4 kHz, in a cochlear region where no synapse loss was seen, and 16 kHz, where the maximum synapse loss occurred. In unexposed animals, the onset-sensitive PSTR peaks display amplitudes that grew with level and varied by frequency. PSTR peak amplitudes were not persistently altered by the noise either within (16 kHz) or outside (4 kHz) the cochlear region maximally injured by the noise. In contrast, the steady-state plateau of the response showed persistent declines at 16 kHz but not 4 kHz.

Although outcomes relative to SR subtype vulnerability after noise can be influenced by the expected frequency/cochlear location of the noise injury and OHC involvement at short post-exposure times, effects on the PSTR plateau isolated to the 16 kHz region, in combination with the complete lack of chronic noise effects on PSTR peak responses at either frequency, together suggest persistent dysfunction of low-SR fibers.

It is widely accepted that sound-evoked gross potentials recorded at the round window are dominated by the response of ANFs populating the cochlear region tuned to the probe frequency. Unitary responses at the gerbil round window appear independent of fiber CF. However, estimating the number of fibers contributing to gross metrics remains difficult.

In control gerbils, the number of synapses (fibers) per IHC was well matched at 4 and 16 kHz (20.8±0.3 at 4 KHz vs. 20.9±0.8 at 16 kHz; FIG. 3C). However, CAP amplitude differences were large (80.8±6 μV at 4 KHz vs. 45.6±2.9 μV at 16 kHz, measured 30 dB above threshold). Similar differences were observed in the PSTR, with a peak of 12.6±1.3 V at 4 kHz vs. 5.2±0.4 μV at 16 KHz and plateau of 1.7±0.2 μV at 4 KHz vs. 0.9±0.1 μV at 16 KHz.

These differences in CAP and PSTR amplitudes at 4 and 16 kHz may be strongly influenced by frequency-dependent differences in the shapes of their respective neural tuning curves. Fibers tuned to 16 KHz display narrower tuning curves than those tuned to 4 kHz, especially evident at lower levels of stimulation (i.e., 30 dB above threshold). Sound stimulation at 16 kHz will therefore recruit a more restricted number of fibers sharply tuned to the probe stimulation than 4-kHz stimulation, which will recruit a larger number of fibers and thus display broader tuning. Fibers tuned to 16 KHz also exhibit higher saturation firing rates compared to 4 kHz-tuned fibers; however, this difference is probably not sufficient to counterbalance the tuning curve effect.

To circumvent these inherent frequency-dependent effects, the inventors normalized the CAP and PSTR amplitudes measured in exposed animals relative to values from unexposed animals. Because noise-induced synaptopathy does not change the neural tuning curve and rate- vs. -intensity functions of remaining fibers, it can be assumed that the decrease of the CAP and PSTR normalized amplitudes can be attributed to synapse loss. CAP amplitudes in the 8-16 KHz region showed persistent declines at 2 wk post-exposure, with some recovery occurring by 4 wk. PSTR peak amplitudes over the same range of frequencies showed non-significant changes from control at both post-exposure times. This onset-dominated response was a poor predictor of synapse survival in these ears. CAP N1-P1 amplitudes fared somewhat better, with outcomes that varied with frequency, and a generally moderate predictive value for synapse loss. In comparison, PSTR plateau, representing activity from a population of ANFs distributed across SR groups, including low-SR fibers, showed persistent, ˜ 40% declines in tonotopically-appropriate regions and correlations with synapse survivals at both 2 and 4 wk after noise.

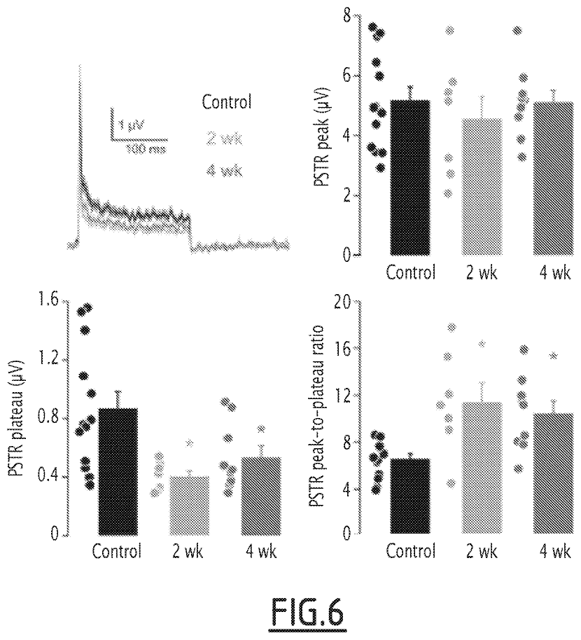

In FIG. 6, the inventors focused on PSTRs obtained at 16 KHz, the region of maximum synaptic loss. Direct comparisons of the PSTR stimulus waveform (FIG. 6 top left), PSTR peak and plateau amplitudes, and their derived peak-to-plateau ratios clearly reveal the sensitivity, particularly of the ratiometric response, in capturing this hidden noise-induced injury (FIG. 6). These parameters of the PSTR are plotted for the 2 wk post-exposure, 4 wk post-exposure, and unexposed control groups. Peak PSTR values (FIG. 6 top right) did not differ significantly among the control and noise-exposed groups. In contrast, significantly smaller PSTR plateaus (FIG. 6 bottom left), driving significantly larger PSTR peak-to-plateau ratios (FIG. 6 bottom right), were seen for both groups of noise-exposed ears. These results suggest a preferential vulnerability of low-SR neurons, which is not well captured by the onset-driven PSTR peak and CAP.

With this experiment, there again, the use of the PSTR appears to be informative as a tool to probe the auditory nerve in etiologies with synaptic and neural compromise.

1. Method for determining at least one parameter of the response of an auditory nerve of a subject, the method being computer-implemented and comprising the following steps:
receiving cochlea response signals, each response signal being the electrical response of a subject's cochlea to a respective excitation, to obtain received response signals,
processing the response signals to obtain a peri-stimulus time response of the auditory nerve, the peri-stimulus time response comprising a peak corresponding to a first amplitude value and a plateau corresponding to a second amplitude value,
calculating the ratio between the first amplitude value and the second amplitude value, to obtain a peak-to-plateau ratio, and
deducing a parameter of the response of the auditory nerve based on the peak-to-plateau ratio.

2. Method for determining according to claim 1, wherein, during the processing step, the received signals are shared into sets of received signals comprising a predefined number of consecutive received signals, preferably two, the processing step comprising:
applying processing operations on each set of received signals, to obtain a set of processed received signals, and
applying an average operation on the set of processed received signals to obtain the peri-stimulus time response of the auditory nerve.

3. Method for determining according to claim 2, wherein the processing operations comprises:
a neurophonic isolation operation to obtain a neurophonic potential, and
an extracting operation, the extracting operation extracting the temporal envelope of the neurophonic potential.

4. Method for determining according to claim 3, wherein the neurophonic isolation comprises an averaging sub-operation followed by a filtering sub-operation.

5. Method for determining according to claim 4, wherein the averaging sub-operation comprises applying a weighted sum.

6. Method for determining according to claim 4, wherein the filtering sub-operation comprises applying a band-pass filter.

7. Method for determining according to claim 6, wherein a lower frequency is defined for the band-pass filter, the lower frequency being comprised between 200 Hz and 700 Hz, preferably between 200 Hz and 400 Hz.

8. Method for determining according to claim 6, wherein an upper frequency is defined for the band-pass filter, the upper frequency being comprised between 1000 Hz and 1500 Hz, preferably between 1100 Hz and 1300 Hz.

9. Method for determining according to claim 3, wherein the extracting operation comprises applying a rectification sub-operation followed by a smoothing sub-operation.

10. Method for determining according to claim 1, wherein each excitation is a bandpass-filtered noise.

11. Method for determining according to claim 1, wherein the method comprises a step for fitting the peri-stimulus time response by two decaying exponential functions to determine the first amplitude value and the second amplitude value.

12. Method for determining according to claim 1, wherein a parameter determined at the determining step is the spontaneous rate of the fibers of the auditory nerve.

13. (canceled)

14. A computer program product comprising instructions for carrying out the steps of a method according to claim 1 when said computer program product is executed on a suitable computer device.

15. A computer readable medium having encoded thereon a computer program according to claim 14.

16. Method for predicting that a subject is at risk of suffering from an auditory disorder, the method for predicting at least comprising the step of:
carrying out the steps of a method determining at least one parameter of the response of an auditory nerve of the subject, to obtain at least one determined parameter, the method for determining being according to claim 1, and
predicting that the subject is at risk of suffering from the auditory disorder based on the determined parameters.

17. Method for diagnosing an auditory disorder, the method for diagnosing at least comprising the step of:
carrying out the steps of a method determining at least one parameter of the response of an auditory nerve of the subject, to obtain at least one determined parameter, the method for determining being according to claim 1, and
diagnosing the auditory disorder based on the determined parameters;

18. Method for identifying a therapeutic target for preventing and/or treating an auditory disorder, the method comprising the steps of:
carrying out the steps of a method determining at least one parameter of the response of an auditory nerve of the first subject, to obtain at least one first determined parameter, the method for determining being according to claim 1 and the first subject being a subject suffering from the auditory disorder, carrying out the steps of a method for determining at least one parameter of the response of an auditory nerve of the second subject, to obtain at least one second determined parameter, the method for determining being according to claim 1 and the second subject being a subject not suffering from the auditory disorder, and
selecting a therapeutic target based on the comparison of the first and second determined parameters;

19. Method for identifying a biomarker, the biomarker being a diagnostic biomarker of an auditory disorder, a prognostic biomarker of an auditory disorder or a predictive biomarker in response to the treatment of an auditory disorder, the method comprising the steps of:
carrying out the steps of a method for determining at least one parameter of the response of an auditory nerve of a first subject, to obtain at least one first determined parameter, the method for determining being according to claim 1 and the first subject being a subject suffering from the auditory disorder,
carrying out the steps of a method for determining at least one parameter of the response of an auditory nerve of a second subject, to obtain at least one second determined parameter, the method for determining being according to claim 1 and the second subject being a subject not suffering from the auditory disorder,
selecting a biomarker based on the comparison of the first and second determined parameters.

20. Method for screening a compound useful as a probiotic, a prebiotic or a medicine, the compound having an effect on a known therapeutical target, for preventing and/or treating an auditory disorder, the method comprising the steps of
carrying out the steps of a method for determining at least one parameter of the response of an auditory nerve of a first subject, to obtain at least one first determined parameter, the method for determining being according to claim 1 and the first subject being a subject suffering from the auditory disorder and having received the compound,
carrying out the steps of the method for determining at least one parameter of the response of an auditory nerve of a second subject, to obtain at least one second determined parameter, the method for determining being according to claim 1 and the second subject being a subject suffering from the auditory disorder and not having received the compound,
selecting a compound based on the comparison of the first and second determined parameters.

21. Method for adjusting a hearing aid or a cochlear implant, the method comprising carrying the steps of a method for determining at least one parameter of the response of an auditory nerve of a subject wearing the hearing aid or the cochlear implant to be adjusted.
